(19) United States
(12) Patent Application Publication
(10) Pub. No.: US 2025/0073373 A1
(43) Pub. Date: Mar. 6, 2025

(54) FREEZE-RESISTANT INJECTABLE SHEAR-THINNING COMPOSITIONS

(71) Applicant: Boston Scientific Scimed, Inc., Maple Grove, MN (US)

(72) Inventors: Nicolas Ball-Jones, Minneapolis, MN (US); Samuel Michael Hanson, Minneapolis, MN (US); Paul Vincent Grosso, Maple Grove, MN (US); Karen Dubbin, Mountain View, CA (US); Gary Thomas Ohrt, Corcoran, MN (US)

(73) Assignee: Boston Scientific Scimed, Inc., Maple Grove, MN (US)

(21) Appl. No.: 18/817,662

(22) Filed: Aug. 28, 2024

Related U.S. Application Data
(60) Provisional application No. 63/579,436, filed on Aug. 29, 2023.

(51) Int. Cl. A61L 27/22; A61L 27/04; A61L 27/54

(52) U.S. Cl. CPC A61L 27/222; A61L 27/047; A61L 27/54; A61L 2400/06

(57) ABSTRACT

The present disclosure pertains to injectable shear-thinning compositions that comprise (a) one or more types of fibrous proteins, (b) one or more types of silicate microparticles, (c) one or more types of radiopaque additives, (d) one or more types of cryoprotectants, and (e) water. Other aspects of the present disclosure pertain to kits that contains such compositions and medical procedures that comprise administering such compositions to a patient.

### CROSS-REFERENCE TO RELATED APPLICATIONS

This application claims the benefit of U.S. Provisional Patent Application Ser. No. 63/579,436 filed on Aug. 29, 2023, the disclosure of which is incorporated herein by reference.

### FIELD

The present disclosure relates to injectable shear-thinning compositions in which freezing is prevented or in which the effects of freezing are mitigated. The injectable shear-thinning compositions are useful, for example, in various medical procedures.

### BACKGROUND

Injectable shear-thinning compositions are attractive due to their minimally invasive delivery procedure, providing reduced healing time, reduced scarring, decreased risk of infection, and ease of delivery compared with surgically implanted materials. Injectable shear-thinning compositions are especially useful for applications where the final form and shape are either not important or are defined by the void or space into which they are injected. Due to their ease of delivery, injectable shear-thinning compositions are potentially useful in a number of areas such as providing a structural or space-filling function, functioning as embolic agents for diverting or eliminating flow in blood vessels, acting as tissue engineering compositions, and delivery of drugs.

One currently available injectable shear-thinning composition is Obsidio™ Conformable Embolic from Boston Scientific Corporation, Marlborough, Massachusetts, USA. It is pre-packaged in a ready-to-use syringe. As the material is pushed through a catheter on its way to a targeted site during administration, the material shear-thins and flows readily, like a liquid. When shear forces are removed as the material reaches its intended location, it reverts to a soft solid that molds to the targeted vasculature's shape, creating a physical barrier that stops blood flow. The Obsidio™ embolic composition contains laponite, gelatin, water and tantalum and is the only conformable embolic indicated to control bleeding and stop blood flow to tumors in the peripheral vasculature. The material, however, requires temperature-controlled shipment. If the material is frozen, the intermolecular structure can fragment and lose cohesion.

### SUMMARY

There is an ongoing need in the biomedical arts for injectable shear-thinning compositions that are stable under low temperature conditions less than 0° C.

In some aspects, the present disclosure pertains to an injectable shear-thinning composition that comprises (a) one or more types of fibrous proteins, (b) one or more types of silicate microparticles, (c) one or more types of radiopaque additives, (d) one or more types of cryoprotectants, and (e) water.

In some embodiments, the one or more types of fibrous proteins are collagen-based proteins. In some of these embodiments, the one or more types of fibrous proteins comprise gelatin.

In some embodiments, which can be used in conjunction with the above aspects and embodiments, the one or more types of silicate microparticles comprise natural and/or synthetic silicate layered clays.

In some embodiments, which can be used in conjunction with the above aspects and embodiments, the one or more types of radiopaque additives comprise radiopaque microparticles that contain radiopaque metals or radiopaque metal compounds. In some of these embodiments, the radiopaque microparticles are tantalum microparticles.

In some embodiments, which can be used in conjunction with the above aspects and embodiments, the injectable shear-thinning composition has a freezing point below −20° C.

In some embodiments, which can be used in conjunction with the above aspects and embodiments, the injectable shear-thinning composition does not phase separate or fragment upon being frozen and then thawed at room temperature.

In some embodiments, which can be used in conjunction with the above aspects and embodiments, the one or more types of cryoprotectants are selected from small molecule cryoprotectants, cryoprotective lipids, cryoprotective surfactants, and cryoprotective polymers.

In some embodiments, which can be used in conjunction with the above aspects and embodiments, the one or more types of cryoprotectants are selected from amino acids, sugars and sugar alcohols.

In some embodiments, which can be used in conjunction with the above aspects and embodiments, the injectable shear-thinning composition further comprises one or more additional agents selected from therapeutic agents, imaging agents, colorants, tonicity adjusting agents, suspension agents, wetting agents, and pH adjusting agents.

In some embodiments, which can be used in conjunction with the above aspects and embodiments, the injectable shear-thinning composition is a sterile composition.

In some embodiments, which can be used in conjunction with the above aspects and embodiments, the injectable shear-thinning composition is provided in a preloaded syringe.

In other aspects, the present disclosure pertains to a kit that comprises one or more containers that contain the injectable shear-thinning composition in accordance with any of the above aspects and embodiments and a delivery device.

In some embodiments, delivery device comprises a syringe, a needle and optionally, a catheter.

In other aspects, the present disclosure pertains to a medical procedure comprising administering to a subject an injectable shear-thinning composition in accordance with any of the above aspects and embodiments.

In some embodiments, the method comprises injecting the injectable shear-thinning composition into the subject.

In some embodiments, which can be used in conjunction with the above aspects and embodiments, the administering comprises parenteral administration.

In some embodiments, which can be used in conjunction with the above aspects and embodiments, the administering is performed using a catheter or a syringe.

In some embodiments, which can be used in conjunction with the above aspects and embodiments, the administering is performed under image guidance.

Potential benefits of the present disclosure include the provision of injectable shear-thinning compositions in which freezing is prevented or in which the effects of freezing are mitigated, when subjected to low temperature conditions less than 0° C.

### BRIEF DESCRIPTION OF THE DRAWINGS

FIG. 1 schematically illustrates a catheter and a syringe that is loaded with an injectable shear-thinning composition, in accordance with an embodiment of the present disclosure.

### DETAILED DESCRIPTION

In some aspects, the present disclosure pertains to injectable shear-thinning compositions that comprise (a) one or more types of fibrous proteins, (b) one or more types of silicate microparticles, (c) one or more types of radiopaque additives, (d) one or more types of cryoprotectant, and (e) water.

A composition is a shear-thinning composition if the viscosity decreases as the shear rate increases. As used herein, a cryoprotectant is a substance that, either alone or in combination with another cryoprotectant, prevents freezing of the shear-thinning compositions described herein or stabilizes the properties of the shear-thinning compositions described herein during freezing and subsequent thawing.

In some embodiments, the injectable shear-thinning compositions of the present disclosure have a freezing point below −20° C. In some embodiments, the injectable shear-thinning compositions of the present disclosure do not phase separate or fragment upon being frozen (for example, upon being exposed to a temperature below −20° C.) and then allowed to warm to room temperature.

In some embodiments, the injectable shear-thinning compositions of the present disclosure have a pH between 6 and 11, more typically between 8 and 10.

In some embodiments, the injectable shear-thinning compositions of the present disclosure are physically crosslinked hydrogel compositions.

In some embodiments, the injectable shear-thinning compositions of the present disclosure are ionically crosslinked hydrogel compositions.

As used herein, a “hydrogel” refers to a hydrated, three-dimensional polymer-containing network.

In some embodiments, the injectable shear-thinning compositions of the present disclosure flow upon application of a pressure greater than a yield stress of the injectable shear-thinning compositions.

Cryoprotectants in accordance with the present disclosure include freezing-point depressants, additives that stabilize protein folding to prevent phase separation upon freezing and thawing, additives that lead to generation of smaller ice crystals upon freezing, osmolytes, carbohydrates, surfactants, and lipids, among other cryoprotectants.

Particular cryoprotectants for use in the present disclosure include (a) freezing point depressants, including organic solvents such as dimethylsulfoxide (DMSO), and alcohols such as ethanol, glycerol, and/or propylene glycol, (b) osmolytes including urea, thiourea, amino acids such as glycine, proline, glutamic acid and alanine (c) carbohydrates, including sugars such as glucose, sucrose, lactose and trehalose, and sugar alcohols, including C₃-C₆ sugar alcohols such as mannitol, sorbitol, erythritol and inositol, (d) additional small molecule (i.e., molecular weight less than 500 Da) cryoprotectants such as acetamide, acetate salts such as ammonium acetate, sodium acetate and potassium acetate, dimethyl sulfone, formamide, trimethylammonium N-oxide, guanidinium salts such as guanidinium chloride and guanidinium bromide, (e) cryoprotective lipids, including polygalactosyldiacylglycerols, (f) cryoprotective surfactants, including polysorbates such as polysorbate 20 (polyoxyethylene (20) sorbitan monolaurate), polysorbate 40 (polyoxyethylene (20) sorbitan monopalmitate), polysorbate 60 (polyoxyethylene (20) sorbitan monostearate), and polysorbate 80 (polyoxyethylene (20) sorbitan monooleate), (g) additional cryoprotective polymers such as, for instance, carboxylated &-poly-L-lysine,

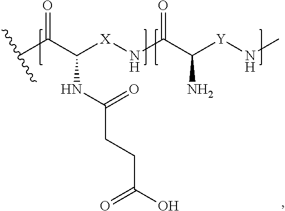

where X and Y are —(CH₂)ₙ— and where n is independently 1, 2, 3, 4, 5, 6, 7, 8 or 9, poly(acrylic acid-co-polydimethylaminoethylmethacrylate),

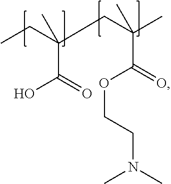

poly([dimethylaminoethyl acrylate-co-acrylic acid-co-polyvinylmethanol),

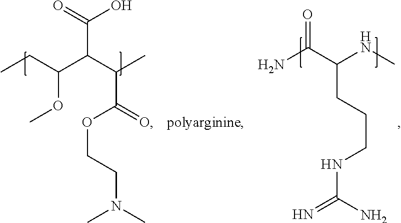

or soy protein isolate, and (h) combinations of any two, three, four, or more of the foregoing.

In some embodiments, the injectable shear-thinning compositions of the present disclosure contain between 0.01 wt % and 20 wt % of the one or more types of cryoprotectants, typically between 2 wt % and 10 wt %.

Fibrous proteins are made up of polypeptide chains that are elongated and fibrous in nature or have a sheet-like structure. Fibrous proteins for use in the injectable shear-thinning compositions of the present disclosure include animal-derived proteins and non-animal-derived proteins. Fibrous proteins for use in the injectable shear-thinning compositions of the present disclosure include keratin, elastin, fibroin, myosin, desmin, fibrin, actin, and collagen, including denatured and hydrolyzed forms thereof such as gelatin. Specific fibrous proteins for use herein include bovine collagen, porcine collagen, equine collagen, porcine gelatin (e.g., type-A porcine gelatin, gelatin derived from porcine skin, gelatin derived from porcine bones, and the like), bovine gelatin (e.g., type-B bovine gelatin, gelatin derived from bovine skin, gelatin derived from bovine bones, and the like), equine gelatin, avian-derived gelatin and fish-derived gelatin.

In some embodiments, the injectable shear-thinning compositions of the present disclosure contain between 0.1 wt % or less and 10 wt % or more of one or more types of fibrous proteins, for example, ranging anywhere from 0.1 wt % to 0.25 wt % to 0.5 wt % to 1 wt % to 2.5 wt % to 5 wt % to 10 wt % (i.e., ranging between any two of the preceding values). In particular embodiments, the injectable shear-thinning compositions of the present disclosure contain between 0.25 wt % and 1 wt % or more of one or more types of fibrous proteins.

Silicate microparticles for use in the injectable shear-thinning compositions of the present disclosure include natural silicate microparticles and synthetic silicate microparticles. Particular examples of silicate microparticles include natural and synthetic silicate layered clays. Natural silicate layered clays include montmorillonite, saponite, hectorite, kaolinite, palygorskite and sepiolite, among others. Synthetic silicate layered clays include lithium magnesium sodium silicates such as Laponite®-based silicate nanoplatelets (e.g., Laponite® XLG-based silicate nanoplatelets, Laponite® XLS-based silicate nanoplatelets, Laponite® XL21-based silicate nanoplatelets, and Laponite® D-based silicate nanoplatelets), Sumecton® SWN and Lucentite™ SWN, magnesium aluminum silicates such as Sumecton® SA, sodium magnesium silicates such as Optigel® SH and SUPLITE-MP, and fluoromica such as Somasif™ ME100, among others.

Silicate microparticles for use in the injectable shear-thinning compositions of the present disclosure may have a size ranging from 5 nm or less to 75 nm or more in longest dimension (e.g., diameter for a sphere, length for a rod, greatest width for a plate-shaped particle, etc.), for example ranging anywhere from 5 nm to 10 nm to 25 nm to 50 nm to 75 nm in longest dimension.

The silicate microparticles for use in the injectable shear-thinning compositions of the present disclosure include microparticles that have a neutral charge, microparticles that have a net positive charge, and microparticles that have a net negative charge. The net charge of the silicate microparticles may depend upon the pH of the injectable shear-thinning composition. In some embodiments, the silicate microparticles have a net positive charge at the pH of the injectable shear-thinning composition. In some embodiments, the silicate microparticles have a negative positive charge at the pH of the of the injectable shear-thinning composition.

In some embodiments, the silicate microparticles are plate-shaped. In some embodiments, the silicate microparticles are silicate layered clays characterized by a discotic charge distribution on the surface. In some embodiments, the plate-shaped silicate microparticles comprise a positively charged edge and a negatively charged surface. In some embodiments, the overall charge of the silicate microparticles is negative. In some embodiments, the plate-shaped silicate microparticles are from about 5 nm to about 60 nm in diameter, for example, from about 10 nm to about 40 nm in diameter, from about 10 nm to about 30 nm in diameter, or from about 20 to about 30 nm in diameter. In some embodiments, the plate-shaped silicate microparticles are from about 0.5 nm to about 2 nm in thickness, or about 1 nm in thickness.

In some embodiments, the injectable shear-thinning compositions of the present disclosure contain between 1 wt % and 15 wt % of one or more types of silicate microparticles, typically between 3 wt % and 6 wt %.

Radiopaque additives for use in the injectable shear-thinning compositions of the present disclosure include radiopaque microparticles that contain radiopaque metals and/or radiopaque metal compounds. Such radiopaque microparticles may be spherical or non-spherical. For example, the radiopaque microparticles may contain a radiopaque metal or metal compound throughout, may contain a cladding of a radiopaque metal or metal compound that surrounds a core of another material such as stainless steel, or may contain a core of a radiopaque metal or metal compound surrounded by a cladding of another material such as stainless steel. Radiopaque metals include transition metals such as tantalum, gold, platinum, tungsten, and alloys containing one or more of the same. Radiopaque metal compounds include compounds of barium, tantalum, bismuth, or gold such as barium sulfate, tantalum oxide, bismuth oxide, bismuth subcarbonate, bismuth oxychloride, gold oxide, tungsten oxide, and tungsten carbide.

Silicate microparticles for use in the injectable shear-thinning compositions of the present disclosure may have a size ranging from 5 nm to 70 microns in longest dimension (e.g., diameter for a sphere, length for a rod, greatest width for a plate-shaped particle, etc.), for example, ranging anywhere from 5 nm to 10 nm to 25 nm to 50 to 100 nm to 250 nm to 500 nm to 1 micron to 2.5 microns to 5 microns to 10 microns to 50 microns to 70 microns.

In some embodiments, the injectable shear-thinning compositions of the present disclosure contain between 10 wt % and 50 wt % of one or more types of radiopaque additives, typically between 15 wt % and 30 wt %.

In various embodiments, the injectable shear-thinning compositions in accordance with the present disclosure have a radiopacity that is greater than 100 Hounsfield units (HU), beneficially ranging anywhere from 100 HU to 250 HU to 500 HU to 750 HU to 1000 HU or more.

The water in the injectable shear-thinning compositions of the present disclosure may be provided in the form of ultrapure water, water for injection, saline, phosphate buffered saline, or high-ion-content water.

In some embodiments, the injectable shear-thinning compositions of the present disclosure contain between 50 wt % and 90 wt % of water, typically between 65 wt % and 85 wt %.

The injectable shear-thinning compositions of the present disclosure may be formed using a variety of methods. The one or more types of fibrous proteins, one or more types of silicate microparticles, one or more types of radiopaque additives, one or more types of cryoprotectants, and water may be mixed in any suitable order. Mixing may be performed by any suitable mixing technique, including, for example, centrifugal mixing, manual mixing, high shear dispersing, vacuum mixing, vortexing, and/or syringe-to-syringe mixing.

The injectable shear-thinning compositions of the present disclosure may be sterilized using any suitable method. For example, the compositions may be autoclaved while inside a reservoir, such as a syringe barrel, vial, or ampule by heating the mixture at or to a temperature of about 121° C. Alternatively or additionally, the compositions may be sterilized via sterile filtration and/or by supercritical CO₂, gamma, x-ray or electron beam irradiation.

In various embodiments, the injectable shear-thinning compositions of the present disclosure may contain one or more agents in addition to one or more types of fibrous proteins, the one or more types of silicate microparticles, one or more types of radiopaque additives, one or more types of cryoprotectants, and the water. Examples of such additional agents include therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, antibodies, anti-cancer drugs, antiproliferative agents, anti-inflammatory agents, hyperplasia inhibiting agents, anti-restenosis agents, steroids, anti-allergic agents, hemostatic agents, smooth muscle cell inhibitors, antibiotics, antimicrobials, anti-fungal agents, analgesics, anesthetics, immunosuppressants, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, and STING (stimulator of interferon genes) agonists, among others.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd^{(III)}, Mn^{(II)}, Fe^{(III)} and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the injectable shear-thinning compositions of the present disclosure, allowing for deep tissue imaging and device marking, for instance, NIR-sensitive nanoparticles such as gold nanoshells, carbon nanotubes (e.g., nanotubes derivatized with hydroxyl or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, and (e) imageable radioisotopes including ^{99m}Tc, ²⁰¹Th, ⁵¹Cr, ⁶⁷Ga, ⁶⁸Ga, ¹¹¹In, ⁶⁴Cu, ⁸⁹Zr, ⁵⁹Fe, ⁴²K, ⁸²Rb, ²⁴Na, ⁴⁵Ti, ⁴⁴Sc, ⁵¹Cr and ¹⁷⁷Lu, among others.

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block copolymers, etc.), among others, and pH adjusting agents including various buffer solutes.

The injectable shear-thinning compositions of the present disclosure may be stored and transported in a sterile form. The injectable shear-thinning compositions may be shipped, for example, in a syringe, catheter, vial, ampoule, or other container.

In various embodiments, kits are provided, which may include one or more containers of shear-thinning compositions as described herein as well other components. For example, the kits may include one or more delivery devices for delivering the injectable shear-thinning compositions to a subject such as syringes, catheters or tubing sets. In some embodiments, the kits may comprise a shear-thinning composition as described herein preloaded in a catheter and/or a syringe barrel and/or in a container such as a vial or ampule. Alternatively or in addition, kits may be provided that include one or more accessory devices such as guidewires. Alternatively or in addition, the kits may be provided that include one or more containers of liquid materials (e.g. contrast agent, sterile water for injection, physiological saline, phosphate buffer, etc.). Alternatively or in addition, the kits may further comprise an additional therapeutic agent, which may be selected, for example, from those described above, among others. Instructions, either as inserts or as labels, indicating quantities of the composition to be administered and/or guidelines for administration can also be included in the kits provided herein. In some embodiments, the instructions comprise instructions for performing one or more of the methods provided herein.

The injectable shear-thinning compositions described herein can be administered by a variety of routes, depending upon the desired medical outcome. In some embodiments, the administering comprises injecting the injectable shear-thinning composition. In some embodiments, the injectable shear-thinning compositions are administered by parenteral administration. Parenteral administration includes intravenous, intraarterial, subcutaneous, intraperitoneal, or intramuscular injection or infusion. In some embodiments, the administering comprises an image guided procedure where computed tomography, fluoroscopy or ultrasound imaging is used to deliver the composition. In some embodiments, the administering comprises injecting the injectable shear-thinning composition into the vascular system of a subject. In some embodiments, the administering comprises injecting the injectable shear-thinning composition into a cancer of the subject or the vasculature supplying a cancer of the subject. In some embodiments, the administering is performed using a catheter or a syringe.

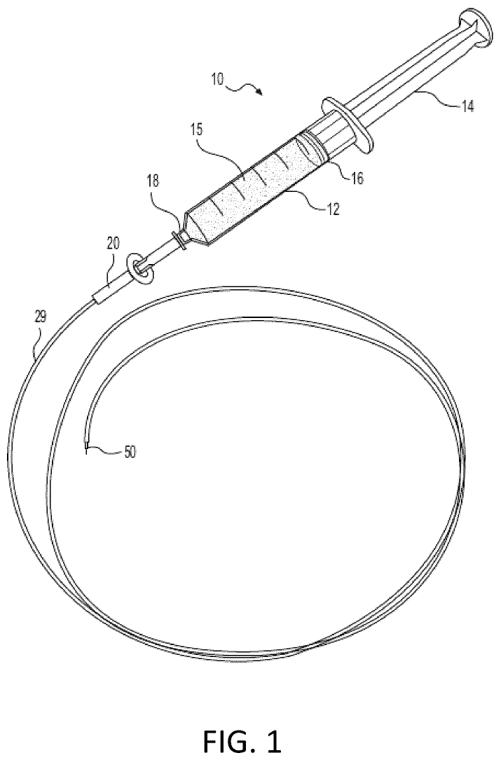

FIG. 1 illustrates an exemplary syringe 10 providing a reservoir for a shear-thinning composition as discussed above. The syringe 10 may comprise a barrel 12, a plunger 14, and one or more stoppers 16. The barrel 12 may include a Luer adapter (or other suitable adapter/connector), e.g., at the distal end 18 of the barrel 12, for attachment to an injection needle 50 via a flexible catheter 29. The proximal end of the catheter 29 may include a suitable connection 20 for receiving the barrel 12. In other examples, the barrel 12 may be directly coupled to the injection needle 50. The syringe barrel 12 may serve as a reservoir, containing a shear-thinning composition 15 for injection through the needle 50.

The injectable shear-thinning compositions described herein can be administered to patients for achieving a number of medical outcomes.

The injectable shear-thinning compositions described herein can be visualized (e.g., within a mammal) using any appropriate method during and/or after administration. For example, imaging techniques such as ultrasound, computed tomography, magnetic resonance imaging, and/or fluoroscopy can be used to visualize the injectable shear-thinning compositions provided herein.

The injectable shear-thinning compositions can be injected to provide spacing between tissues, the injectable shear-thinning compositions can be injected (e.g., in the form of blebs) to provide fiducial markers, the injectable shear-thinning compositions can be injected for tissue augmentation or regeneration, the injectable shear-thinning compositions can be injected as a filler or replacement for soft tissue, the injectable shear-thinning compositions can be injected to provide mechanical support for compromised tissue, the injectable shear-thinning compositions can be injected as a scaffold, and/or the injectable shear-thinning compositions can be injected as a carrier of therapeutic agents in the treatment of diseases and cancers and the repair and regeneration of tissue, among other uses.

The injectable shear-thinning compositions of the present disclosure may be used in a variety of medical procedures, including the following, among others: a procedure to implant a fiducial marker comprising the injectable shear-thinning compositions, a procedure to implant a tissue regeneration scaffold comprising the injectable shear-thinning compositions, a procedure to implant a tissue support comprising the injectable shear-thinning compositions, a procedure to implant a tissue bulking agent comprising the injectable shear-thinning compositions, a procedure to implant a therapeutic-agent-containing depot comprising the injectable shear-thinning compositions, a tissue augmentation procedure comprising implanting the injectable shear-thinning compositions, a procedure to embolize tissue, including benign tumors, malignant tumors and other abnormal tissue, a procedure to control bleeding, a procedure to introduce the injectable shear-thinning compositions between a first tissue and a second tissue to space the first tissue from the second tissue.

The injectable shear-thinning compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

The injectable shear-thinning compositions may be injected for the permanent or temporary occlusion of blood vessels, and thus may be useful for managing various diseases and conditions. For example, the injectable shear-thinning compositions may be used for the controlled, selective obliteration of the blood supply to benign and malignant tumors including treating solid tumors such as renal carcinoma, bone cancer, brain cancer, liver cancer, breast cancer, prostate cancer, benign prostatic hyperplasia, esophageal cancer, colon cancer, endometrial cancer, bladder cancer, cancer of the uterus, uterine fibroids (leiomyoma), cancer of the ovary, lung cancer, sarcoma, pancreatic cancer, and stomach cancer. The idea behind this treatment is that the flow of blood, which supplies nutrients to the tumor, will be blocked causing it to shrink. Embolization may be conducted as an enhancement to chemotherapy or radiation therapy. Treatment may be enhanced in the present disclosure by including a therapeutic agent (e.g., antineoplastic/antiproliferative/anti-miotic agent, toxin, ablation agent, etc.) in the particulate composition.

Shear-thinning compositions in accordance with the present disclosure may also be used to treat various other diseases, conditions and disorders, including treatment of the following: arteriovenous fistulas and malformations including, for example, aneurysms such as neurovascular and aortic aneurysms, pulmonary artery pseudoaneurysms, intracerebral arteriovenous fistula, cavernous sinus, dural arteriovenous fistula and arterioportal fistula, varices, chronic venous insufficiency, varicocele, abscesses, pelvic congestion syndrome, gastrointestinal bleeding, renal bleeding, urinary bleeding, varicose bleeding, venous congestion disorder, hemorrhage, including uterine hemorrhage, and severe bleeding from the nose (epistaxis), as well as preoperative embolization (to reduce the amount of bleeding during a surgical procedure) and occlusion of saphenous vein side branches in a saphenous bypass graft procedure, among other uses. As elsewhere herein, treatment may be enhanced in the present disclosure by including a therapeutic agent in the particulate composition.

Shear-thinning compositions in accordance with the present disclosure may be used further in tissue bulking applications, for example, as augmentative materials in the treatment of urinary incontinence, vesicourethral reflux, fecal incontinence, intrinsic sphincter deficiency (ISD) or gastro-esophageal reflux disease, or as augmentative materials for aesthetic improvement. For instance, a common method for treating patients with urinary incontinence is via periurethral or transperineal injection of a bulking material. In this regard, methods of injecting bulking agents commonly require the placement of a needle at a treatment region, for example, periurethrally or transperineally. The bulking agent is injected into a plurality of locations, assisted by visual aids, causing the urethral lining to coapt. In some cases, additional applications of bulking agent may be required. Treatment may be enhanced by including a therapeutic agent (e.g., proinflammatory agents, sclerosing agents, etc.) in the particulate composition.

Although various embodiments are specifically illustrated and described herein, it will be appreciated that modifications and variations of the present disclosure are covered by the above teachings and are within the purview of any appended claims without departing from the spirit and intended scope of the present disclosure.

What is claimed is:

1. An injectable shear-thinning composition comprising (a) one or more types of fibrous proteins, (b) one or more types of silicate microparticles, (c) one or more types of radiopaque additives, (d) one or more types of cryoprotectants, and (e) water.

2. The injectable shear-thinning composition of claim 1, wherein the one or more types of fibrous proteins are collagen-based proteins.

3. The injectable shear-thinning composition of claim 2, wherein the one or more types of fibrous proteins comprise gelatin.

4. The injectable shear-thinning composition of claim 1, wherein the one or more types of silicate microparticles comprise natural and/or synthetic silicate layered clays.

5. The injectable shear-thinning composition of claim 1, wherein the one or more types of radiopaque additives comprise radiopaque microparticles that contain radiopaque metals or radiopaque metal compounds.

6. The injectable shear-thinning composition of claim 5, wherein the radiopaque microparticles are tantalum microparticles.

7. The injectable shear-thinning composition of claim 1, wherein the injectable shear-thinning has a freezing point below −20° C.

8. The injectable shear-thinning composition of claim 1, wherein the injectable shear-thinning composition does not phase separate or fragment upon being frozen and then thawed at room temperature.

9. The injectable shear-thinning composition of claim 1, wherein the one or more types of cryoprotectants are selected from small molecule cryoprotectants, cryoprotective lipids, cryoprotective surfactants, and cryoprotective polymers.

10. The injectable shear-thinning composition of claim 1, wherein the one or more types of cryoprotectants are selected from amino acids, sugars and sugar alcohols.

11. The injectable shear-thinning composition of claim 1, wherein the injectable shear-thinning composition further comprises one or more additional agents selected from therapeutic agents, imaging agents, colorants, tonicity adjusting agents, suspension agents, wetting agents, and pH adjusting agents.

12. The injectable shear-thinning composition of claim 1, wherein the injectable shear-thinning composition is a sterile composition.

13. The injectable shear-thinning composition of claim 1, wherein the injectable shear-thinning composition is provided in a preloaded syringe.

14. A kit comprising a delivery device and one or more containers that contain an injectable shear-thinning composition that comprises (a) one or more types of fibrous proteins, (b) one or more types of silicate microparticles, (c) one or more types of radiopaque additives, (d) one or more types of cryoprotectants, and (e) water.

15. The kit of claim 14, wherein the delivery device comprises a syringe, a needle and optionally, a catheter.

16. A medical procedure comprising administering to a subject an injectable shear-thinning composition that comprises (a) one or more types of fibrous proteins, (b) one or more types of silicate microparticles, (c) one or more types of radiopaque additives, (d) one or more types of cryoprotectants, and (e) water.

17. The medical procedure of claim 16, wherein the method comprises injecting the injectable shear-thinning composition into the subject.

18. The medical procedure of claim 16, wherein the administering comprises parenteral administration.

19. The medical procedure of claim 16, wherein the administering is performed using a catheter or a syringe.

20. The medical procedure of claim 16, wherein the administering is performed under image guidance.
